(19) United States
(12) Patent Application Publication
(10) Pub. No.: US 2017/0348381 A1
(43) Pub. Date: Dec. 7, 2017

(54) FORMULATION

(71) Applicant: NovaBiotics Limited, Aberdeen (GB)

(72) Inventor: Deborah O'NEIL, Aberdeen (GB)

(21) Appl. No.: 15/614,246

(22) Filed: Jun. 5, 2017

Related U.S. Application Data
(60) Provisional application No. 62/346,959, filed on Jun. 7, 2016.

(51) Int. Cl. A61K 38/10; A61K 31/7048; A61K 31/496; A61K 9/00; A61K 31/145; A61K 9/08; A61K 38/08; A61K 31/506

(52) U.S. Cl. CPC A61K 38/10; A61K 38/08; A61K 31/7048; A61K 31/506; A61K 9/0078; A61K 31/145; A61K 9/08; A61K 31/496

(57) ABSTRACT

The present disclosure relates to an aqueous composition of an antifungal peptide, to methods of treatment using the composition and to uses of the composition.

### CROSS-REFERENCE TO RELATED APPLICATIONS

The present application claims the benefit of U.S. Provisional Application No. 62/346,959, filed on Jun. 7, 2016, the disclosures of which are incorporated herein by reference in their entirety for all purposes.

### FIELD OF THE INVENTION

The present invention relates to an aqueous nebulisable composition comprising an antifungal peptide and water. It further relates to methods of treatment employing the composition and to use of the composition in the treatment or prevention of local and optionally systemic microbial infections.

### BACKGROUND TO THE INVENTION

Fungal infections of the lung often affect the immunocompromised and are common nosocomial infections. Due to the difficulty in administering antifungal agents in nebulised form, the treatment of fungal infections of the lung is notoriously difficult, requiring systemic agents. However, the toxicity of antifungals when used to treat a systemic infection is a cause for concern. The use of direct local treatment via aerosolised/nebulised delivery is an attractive option in prevention and treatment of such infections because the drug can concentrate locally at the site of infection with minimal systemic exposure.

Aspergillosis is a common life threatening condition caused by fungal infection of the lung in the immunocompromised. It presents as both a chronic and invasive local infection of the lungs and as an invasive disseminated fungal infection. It is extremely difficult to treat, not only because of the site of infection but because of difficulties targeting eukaryotic microbes in a eukaryotic host (e.g. similarities in cell membrane etc).

Antifungal agents are typically difficult to nebulise in an aqueous formulation due to being large, hydrophobic molecules. Those antifungal agents that have been nebulised tend to be in liposomal or aerosolised formulations (Le and Schiller, 2010; Castagnola et al 2007).

Clinical evidence for the use of aerosolised delivery in preventing fungal infections is currently limited to amphotericin B products, although itraconazole, voriconazole, and caspofungin are under investigation. However, conflicting results from clinical trials that evaluated various amphotericin B formulations have led to the routine use of aerosolised delivery not being recommended. Thus there is a need to provide a nebulisable antifungal agent that can treat and prevent local fungal infections of the lung.

Novamycin is a known antifungal agent with a fungicidal mode of action. The peptide punctures the fungal cell membrane, lysing the cell, yet has no action against mammalian cells. This mode of action makes the risk of resistance developing extremely low. Novamycin is effective as a systemic drug to address disseminated infections. However, due to the physical properties of the peptide, the present Inventors have surprisingly established that the drug can be successfully nebulised in an aqueous formulation.

### SUMMARY OF THE INVENTION

According to a first aspect, there is provided an aqueous nebulisable composition comprising an antifungal peptide wherein the antifungal peptide is present at a concentration of 0.1 to 100 mg/ml.

The aqueous nebulisable composition may comprise an antifungal peptide and water wherein the antifungal peptide is present at a concentration of 0.1 to 100 mg/ml.

Advantageously, an aqueous nebulisable formulation or composition is simple to administer in a clinical setting because it can be used with standard equipment.

In a second aspect, there is provided a nebulised aqueous composition according to the disclosure.

In a third aspect, there is provided a method of treating a microbial, such as a fungal, infection in a subject comprising administering to the subject an aqueous nebulisable composition according to the disclosure.

Advantageously, administration of a composition by nebuliser allows the antifungal agent to be targeted to the site of infection (i.e. the lung) where is can work directly on the infective microbe. Beneficially, this mode of administration is effective as a preventative or prophylactic measure.

In a fourth aspect, there is provided an aqueous nebulisable composition according to the disclosure for use in the treatment of a local and optionally disseminated microbial infection.

In a fifth aspect, there is provided an aqueous nebulisable composition according to the disclosure for use in the treatment by oral (inhaled) administration once to four times per day, of a local and optionally disseminated microbial infection.

In a sixth aspect, there is provided a kit of parts comprising a composition according to the disclosure and a systemic antimicrobial agent.

In a seventh aspect, there is provided a process of making an aqueous composition according to the disclosure comprising the step of adding an antifungal peptide to water in the amount of 0.1 to 100 mg/ml.

In an eighth aspect, there is provided a nebuliser comprising an aqueous composition according to the disclosure.

In a ninth aspect there is provided a 13 to 15 amino acid linear poly-arginine peptide and an antifungal agent.

### BRIEF DESCRIPTION OF THE DRAWINGS

The present invention will now be described by way of example only with reference to the accompanying Figures in which:

FIG. 1 shows lung tissue burden group average in CFU/g of tissue following treatment with nebulised Novamycin at 1, 5 and 10 mg/ml bidaily versus ambisome at 5 mg/kg once daily further including controls.

FIG. 2 shows a scatterplot of lung tissue burden results from FIG. 1 in CFU/g of tissue following treatment with nebulised Novamycin at 1, 5 and 10 mg/ml bidaily versus ambisome at 5 mg/kg once daily further including controls.

FIG. 3 shows a second in vivo data set lung tissue burden group average in CFU/g of tissue following treatment with nebulised Novamycin at 1, 5 and 10 mg/ml bidaily versus ambisome at 5 mg/kg once daily.

FIG. 4 shows a scatterplot of lung tissue burden results from FIG. 3 in CFU/g of tissue following treatment with nebulised Novamycin at 1, 5 and 10 mg/ml bidaily versus ambisome at 5 mg/kg once daily.

FIG. 5 shows % survival data at various time points post infection following treatment with nebulised Novamycin at 1, 5 and 10 mg/ml bidaily versus ambisome at 5 mg/kg once daily further including controls.

FIG. 6 shows the terminal Aspergillus burden following treatment with nebulised Novamycin at 1, 5 and 10 mg/ml bidaily versus ambisome at 5 mg/kg once daily further including controls.

FIG. 7 shows a more spread out version of the data in FIG. 6, for clarity.

FIG. 8 shows % survival data at various time points following treatment with nebulised novamycin at 1 and 5 mg/ml daily versus IV (systemic) ambisome at 5 mg/kg once daily versus combination therapy of nebulised novamycin at 1 and 5 mg/ml bidaily PLUS ambisome at 5 mg/kg once daily and controls.

### DETAILED DESCRIPTION

Aqueous as employed herein means the composition is primarily aqueous, that is, it contains water. Typically, the composition is primarily water and active ingredient (i.e. antifungal agent). In one embodiment the composition does not contain any excipients. In one embodiment the composition consists of water and at least one antifungal agent, such as an antifungal peptide.

Nebulisable as employed herein means to convert a liquid into a fine spray. Typically, nebulised liquids are inhaled over a prolonged period. That is, they are not inhaled in just one or two breaths in the way an inhaler-dispensed composition would be.

Peptide as employed herein means a therapeutically active peptide or a salt thereof. Typically, the peptide is an antifungal peptide with activity against fungal infections of the lung. The term “peptide” as used herein means, in general terms, a plurality of amino acid residues joined together by peptide bonds. It is used interchangeably and means the same as polypeptide and protein.

In one embodiment the peptide is a linear peptide. That is, the peptide has free ends and is typically not a branched peptide. In one embodiment the peptide is a branched linear peptide.

In one embodiment the peptide comprises approximately 5 to 15 amino acids, such as 6, 7, 8, 9, 10, 11, 12, 13, 14 or 15 amino acids. For example, approximately 10 to 15 amino acids or approximately 14 amino acids, such as 13, 14 or 15 amino acids.

In one embodiment the peptide consists of 13 to 15 amino acids, such as 14 or 15 amino acids.

In one embodiment the number of amino acid residues referred to in the ranges above does not include the histidine tag residues. Thus, in one aspect, histidine residues at either end of the peptide are discounted when determining the numbering of amino acids in the modified peptide. In another embodiment, all amino acid residues are counted including those making up a histidine tag.

In one embodiment, the peptide comprises consecutive amino acids according to the formula (I)

(X)ₙ (I)

wherein X is arginine and n is an integer between 5 and 15.

In one embodiment the peptide is a poly-arginine peptide. That is, a peptide consisting essentially of arginine residues. In one embodiment the peptide is a linear peptide consisting of 14 arginine residues or a pharmaceutically acceptable salt thereof

Modified peptides also encompassed by the present invention. Modified peptides as employed herein means a peptide which has 5 to 15 amino acid residues predominantly arginine further comprising: a histidine tag; and/or a fatty acid and/or a pegylated peptide. Suitably, the modified peptides of the present invention, may be linear peptides. Modified peptides are described in WO2015150823, the disclosure is incorporated herein by reference for all purposes.

The peptides of the invention may generally be synthetic peptides. The peptides may be isolated, purified peptides or variants thereof, which can be synthesised in vitro, for example, by a solid phase peptide synthetic method, by enzyme catalysed peptide synthesis or with the aid of recombinant DNA technology.

To identify active peptides that have little or no undesired toxicity for mammalian cells, individual peptides, or libraries of peptides, can be made and the individual peptides or peptides from those libraries can be screened for antimicrobial activity and toxicity, including, but not limited to, antifungal, antibacterial, antiviral, antiprotozoal, anti-parasitic activity and toxicity.

The peptides of the invention can exist in different forms, such as free acids, free bases, esters and other prodrugs, salts and tautomers, for example, and the invention includes all variant forms of the compounds.

Thus, the invention encompasses the salt or pro-drug of a peptide or peptide variant of the invention.

The peptide of the invention may be administered in the form of a pharmaceutically acceptable salt. The pharmaceutically acceptable salts of the present invention can be synthesized from the parent peptide which contains a basic or acidic moiety by conventional chemical methods. Generally, such salts can be prepared by reacting the free acid or base forms of the peptide with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The invention thus includes pharmaceutically-acceptable salts of the peptide of the invention wherein the parent compound is modified by making acid or base salts thereof for example the conventional non-toxic salts or the quaternary ammonium salts which are formed, e.g., from inorganic or organic acids or bases. Examples of such acid addition salts include, but are not limited to, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include, but are not limited to, ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts of carboxyl groups of a peptide or peptide variant of the invention may be prepared in the usual manner by contacting the peptide with one or more equivalents of a desired base such as, for example, a metallic hydroxide base, e.g. sodium hydroxide; a metal carbonate or bicarbonate such as, for example, sodium carbonate or bicarbonate; or an amine base such as, for example, triethylamine, triethanolamine and the like.

The invention includes prodrugs for the active pharmaceutical species of the described peptide, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular structures which are rapidly transformed in vivo to the parent structure, for example, by hydrolysis in blood.

Suitably, the arginine residue is the predominant amino acid in the peptide. Suitably, at least 50% of the amino acid residues are arginine residues, in some embodiments, at least 60% or at least 70% or at least 80% of the amino acids in the peptide are arginine. In certain embodiments, at least 90% are arginine residues. In some embodiments all the amino acids in the peptide are arginine residues (optionally with the exception of a histidine tag).

Suitably, the peptide may comprise amino acids other than arginine is non-predominant amounts. For example, histidine, ornithine and lysine could be used.

Suitably, 5 to 15 (for instance, contiguous) D and/or L amino acids consist of arginine or a combination of arginine and lysine residues except for 0, 1, or 2 substitutions to an amino acid residues other than arginine or lysine. In some embodiments, such substitutions (if present) are with another cationic amino acids selected from the group consisting of histidine, ornithine and lysine. In certain embodiments, the substitutions are with lysine.

Suitably, the peptide may be substituted with 0, 1, 2, 3, 4, 5, 6 or 7 substitutions provided that the arginine make up at least 60%, in some instances, at least 75% of the peptide.

In some embodiments, the amino acids are L-amino acids.

In one aspect of the invention, at least 90%, for example at least 95% such as 97-99% or even 100%, of the amino acids in the peptide are L-amino acids.

The invention also includes known isomers (structural, stereo-, conformational & configurational), peptidomimetics, structural analogues of the above amino acids, and those modified either naturally (e.g. post-translational modification) or chemically, including, but not exclusively, phosphorylation, glycosylation, sulfonylation and/or hydroxylation.

One or more of the residues of the peptide can be exchanged for another to alter, enhance or preserve the biological activity of the peptide. Such a variant can have, for example, at least about 10% of the biological activity of the corresponding non-variant peptide. Conservative amino acids are often utilised, i.e. substitutions of amino acids with similar chemical and physical properties as described above. Hence, for example, conservative amino acid substitutions may involve exchanging lysine for arginine, ornithine or histidine; or exchanging arginine for lysine or isoleucine, ornithine for histidine; or exchanging one hydrophobic amino acid for another. After the substitutions are introduced, the variants are screened for biological activity.

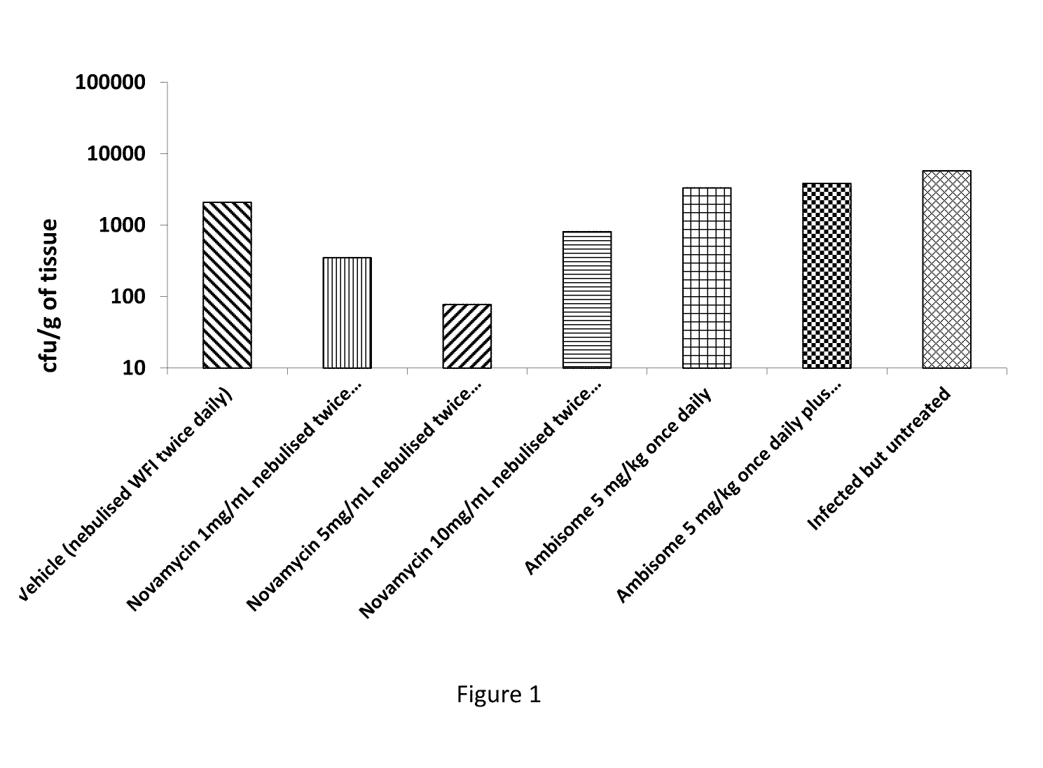

In one embodiment, the composition of the disclosure comprises approximately 0.001-500 mg/ml of peptide, such as approximately 0.1-100 mg/ml of peptide. For example, 0.2, 0.3, 0.4, 0.5, 0.6, 0.7, 0.8, 0.9, 1.0, 1.5, 2.0, 2.5, 5, 10, 15, 20, 25, 30, 35, 40, 45, 50, 55, 60, 65, 70, 75, 80, 85, 90 or 95 mg/ml of peptide. For example, the composition comprises approximately 1-10 mg/ml, and in some instances, approximately 5 mg/ml of peptide. A dose of 5 mg/ml bidaily provided the lowest tissue burden in in vivo studies (see FIG. 1).

In one embodiment, the composition further comprises an antifungal agent.

Also contemplated are combination products that include one or more peptides of the present invention and one or more other antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein.

In one embodiment, the systemic antimicrobial agent is selected from the group consisting of: polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole; glucan synthase inhibitors such as caspofungin, micafungin (FK463), and V-echinocandin (LY303366); griseofulvin; allylamines such as terbinafine; flucytosine or peptides such as Novamycin.

Novamycin as employed herein refers to 14 amino acid linear poly-arginine peptide.

In one embodiment, the antimicrobial or antifungal agent is cysteamine or a derivative thereof such as cystamine.

In one embodiment, the antimicrobial or antifungal agent is ambisome or amphotericin B.

In one embodiment, the antimicrobial or antifungal agent is caspofungin.

In one embodiment, the antimicrobial or antifungal agent is Novamycin.

Method of treating or preventing as employed herein means that a disease or condition maybe treated with the aim of curing or delaying progress the disease or condition or that the composition may be given prophylactically to prevent infection occurring or becoming established. The term “treatment” relates to the effects of the peptides described herein that in imparting a benefit to patients afflicted with an (infectious) disease, including an improvement in the condition of the patient or delay in disease progression.

In one embodiment, the invention provides a method of treating or preventing a fungal infection in a subject comprising administering to said subject a therapeutically effective amount of a peptide according to the invention.

Therapeutically effective amount as employed herein means the amount of active substance, such as antifungal agent, that is administered within the scope of sound medical judgement, is sufficient to provide a desired effect without toxicity, irritation, allergic reaction or other problem or complication commensurate with a reasonable risk/benefit ratio.

In one embodiment, the composition comprises a therapeutically effective amount of at least one peptide.

In one embodiment, the composition comprises a therapeutically effective amount of at least one antifungal agent.

A subject as employed herein means a human or animal subject in need of treatment with compositions of the disclosure.

Mammals, birds and other animals may be treated by the peptides, compositions or methods described herein. Such mammals and birds include humans, dogs, cats and livestock, such as horses, cattle, sheep, goats, chickens and turkeys and the like.

In one embodiment, the method of treatment further comprises simultaneous, sequential or separate administration of a systemic antimicrobial agent.

Simultaneous, sequential or separate administration as employed herein means that the nebulised composition and the systemic antimicrobial agent are administered at the same time, immediately one following the other or at a time spaced apart, such as several minutes to hours apart.

Systemic antimicrobial agent as employed herein means an antimicrobial agent which is administered such that it enters the circulatory system. The method of administration may be enteral (i.e. by absorption from the intestinal tract) or parenteral (e.g. via injection, infusion or implantation).

The antimicrobial agent is useful, inter alia, against bacteria, fungi, yeast, parasites, protozoa and viruses. The term, “antimicrobial agent” can be used herein to define any peptide that has microbicidal and/or microbistatic activity and encompasses, non-exclusively, any agent described as having anti-bacterial, anti-fungal, anti-mycotic, anti-parasitic, anti-protozoal, anti-viral, anti-infectious, anti-infective and/or germicidal, algicidal, amoebicidal, microbicidal, bacterici(o)dal, fungicidal, parasiticidal, protozoacidal, protozoicidal properties.

In one embodiment the fungal infection may be an infection by one or more of the group consisting of: Candida spp., (e.g. C.albicans), Epidermophyton spp., Exophiala spp., Microsporum spp., Trichophyton spp., (e.g T.rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

In one embodiment, the fungal infection is aspergillosis.

In one embodiment, the fungal infection is candidiasis.

In one embodiment, the present invention provides a method of treating or preventing any one or more of the group consisting of: candidiasis (including oropharyngeal candidiasis or OPC), aspergillosis (including bronchopulmonary aspergillosis, chronic pulmonary aspergillosis and aspergillomata), athlete's foot; basidiodiabolomycosis; blastomycosis; coccidioidomycosis cryptoccocis; basal meningitis; dermatophytosis; onchomycosis; dermatophytids; endothrix; exothrix; fungal meningitis, fungemia, histoplasmosis, mycosis, myrinogmycosis, paracoccidioidomycosis, penicilliosis, piedra, pneumocytosis, pneumocystis, pneumonia, sporptrichosis, tinea, zeospora and zygomycosis in a subject, said method comprising administering a pharmaceutically effective amount of the modified peptide of the present invention or a pharmaceutical composition of the present invention. Suitably, the fungal infection may be a Candida infection and/or an Aspergillus infection. Suitably, the administration route may be inhaled.

Suitably in the methods of the present invention the subject may have HIV or AIDS.

The peptides of the composition are potent antifungal peptides for a wide variety of pathogenic yeast and moulds. However, the peptides of the invention may also be useful in the treatment of other conditions including, but not limited to, conditions associated with mucosal infections, for example, cystic fibrosis, gastrointestinal, urogenital, urinary (e.g kidney infection or cystitis) or respiratory infections.

Administration of the composition in accordance with the present disclosure may be in a single dose, in multiple doses, in a continuous or intermittent manner, depending, for example, upon the recipient's physiological condition, whether the purpose of the administration is therapeutic or prophylactic, and other factors known to skilled practitioners. The administration of the composition may be essentially continuous over a pre-selected period of time or may be in a series of spaced doses. Both local and systemic administration is contemplated.

In one embodiment, the composition is administered by oral administration once to four times per day. Such as once, twice, three times or four times daily. For example, a dosage of 0.1-100 mg/ml may be administered once to four times daily for 10 to 60 minutes each dose.

Typically, administration by nebuliser occurs for approximately 10 to 60 minutes, such as approximately 15, 20, 25, 30, 35, 40, 45, 50 or 55 minutes.

In one embodiment, 1-10 mg/ml is administered for up to 30 minutes. In one embodiment 1-10 mg/ml is administered for 20 minutes, for example by nebuliser. For example, administration occurs once to four times daily, such a bidaily.

In one embodiment, 5 mg/ml is administered twice daily.

In one embodiment, the administration is for approximately 20 minutes, such as 20 minutes bidaily.

In one embodiment, 5 mg/ml is administered for approximately 20 minutes bidaily.

In one embodiment, oral administration means by nebulisation, that is, inhaled administration.

As employed herein oral administration refers to inhaled administration.

Typically, administration occurs for 1 to 30 days such as 2, 3, 4, 5, 6, 7, 8, 9, 10, 11, 12, 13, 14, 15, 16, 17, 18, 19, 20, 21, 22, 23, 24, 25, 26, 27, 28 or 29 days. For example, administration occurs for up to 20 days. Typically, administration is for approximately 14 days.

In one embodiment, administration is repeated for up to 20 days, such as for 14 days.

The term, “approximately” as employed herein means ±10%.

Local microbial infection as employed herein means an infection that is concentrated in one location. That is, the infection is not disseminated or systemic.

In one embodiment, the local infection is in the lung.

Disseminated microbial infection as employed herein means that an infection has extended beyond its origin or nidus and involved the circulatory system to “seed” other areas of the body. As an example, one can view metastatic cancer as a disseminated condition in that it has extended into the bloodstream or into the lymphatic system and thus “seeded” distant sites.

Compositions of the disclosure may further comprise carriers and/or diluents. Specific non-limiting examples of the carriers and/or diluents that are useful in the pharmaceutical formulations of the present invention include water and physiologically acceptable buffered saline solutions such as phosphate buffered saline solutions pH 7.0-8.0.

The peptides of the invention are typically administered to the respiratory tract. For instance, administration can be by inhalation or insufflation. Compositions of the present invention are administered in an aqueous solution when administered in an aerosol or inhaled form. Thus, other aerosol pharmaceutical formulations may comprise, for example, a physiologically acceptable buffered saline solution containing between about 0.001 mg/ml and about 500 mg/ml for example between 0.1 and 100 mg/ml, such as 0.5-50 mg/ml, 0.5-20 mg/ml, 0.5-10 mg/ml, 0.5-5 mg/ml or 1-5 mg/ml of antifungal peptide specific for the indication or disease to be treated.

In one embodiment, the antifungal agent is selected from the group consisting of: cysteamine, Novamycin, amphotericin B, caspofungin, pozaconazole, itraconazole and fluconazole.

In one embodiment, there is provided simultaneous, sequential or separate inhaled and systemic administration of Novamycin.

In one embodiment, the composition further comprises inhaled cysteamine. In one embodiment the cysteamine may be administered simultaneously, sequentially or separately.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### EXAMPLES

### Example 1

### Chemicals

Ceftriaxone, cyclosphosphamide and cortisone acetate were prepared for immunosuppression and pre-conditioning of animals at 50 mg/kg, 150 mg/kg and 175 mg/kg respectively. Novamycin was prepared for twice daily nebulised dosing at 1 and 5 mg/ml, and Ambisome prepared for once daily IV dosing at 5 mg/kg. Phosphate buffered saline (PBS) and Sabouraud dextrose agar containing 50 μg/ml chloramphenicol were required for fungal tissue burden.

### Animals

Male CD1 mice (n=8 for treatment groups, plus four uninfected controls, totalling 60 mice in total) were used in the study. On days −4 to +1, the mice were immunosuppressed/pre-conditioned with 50 mg/kg ceftriazone sub-cutaneously; and on days −4 to −1 they were immunosuppressed/pre-conditioned with 150 mg/kg cyclosphosphamide intraperitoneally. In addition, on day −1 mice were treated with 175 mg/kg cortisone acetate sub-cutaneously. Anaesthetised mice were infected intranasally with 0.04 ml of an inoculum of 1.8×10⁶ cfu/ml Aspergillus fumigatus A1163 (7.2×10⁴ cfu/animal) which established a robust infection in the lungs.

### Treatment

Nebulised Novamycin was administered twice daily at 1 and 5 mg/ml alone and in combination with Ambisome IV at 5 mg/kg once daily. Additional groups included 5 mg/kg Ambisome IV once daily plus nebulised vehicle, infected but untreated controls, and treated but uninfected controls. The study lasted four days post-infection.

### Fungal Burden in Mouse Lung

The lung tissue burden at the clinical end point of each animal was determined; following euthanasia, the lungs are removed and weighed. Lung samples are homogenised in 2 ml ice cold sterile phosphate buffered saline. Organ homogenates are quantitatively cultured following serial dilution on to Sabouraud dextrose agar containing 50 μg/ml chloramphenicol and incubated at 37° C. for up to 4 days and colonies counted. In addition, serum samples and a sample of lung homogenate were taken for Galactomannan and PCR analysis. All uninfected treatment control mice survived to 96 h. Within the treatment groups two out of the eight mice treated with 5 mg/ml Novamycin+5 mg/kg Ambisome, and one of the eight mice treated with 1 mg/kg Novamcyin+5 mg/kg Ambisome survived to 96 hours. Within all the other treatment groups, all the mice succumbed to the infection before the end of the study. The lung tissue burdens did not differ significantly between the groups, however the Galactomannan analysis of serum samples using the PlateliaTM Aspergillus Ag Galactomannan EIA assay kit showed that the mice treated with 5 mg/ml Novamycin +5 mg/kg Ambisome had the lowest Galactomannan index reflecting the survival results.

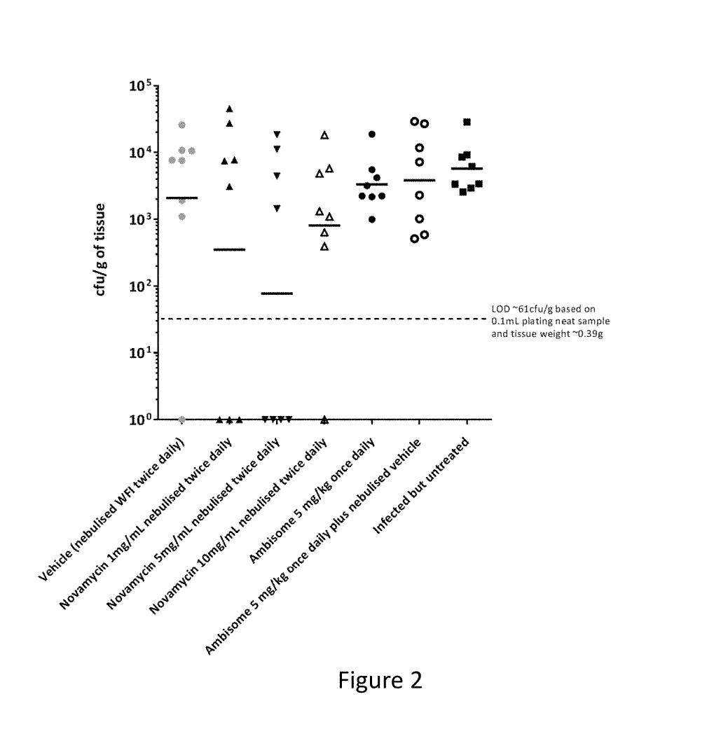

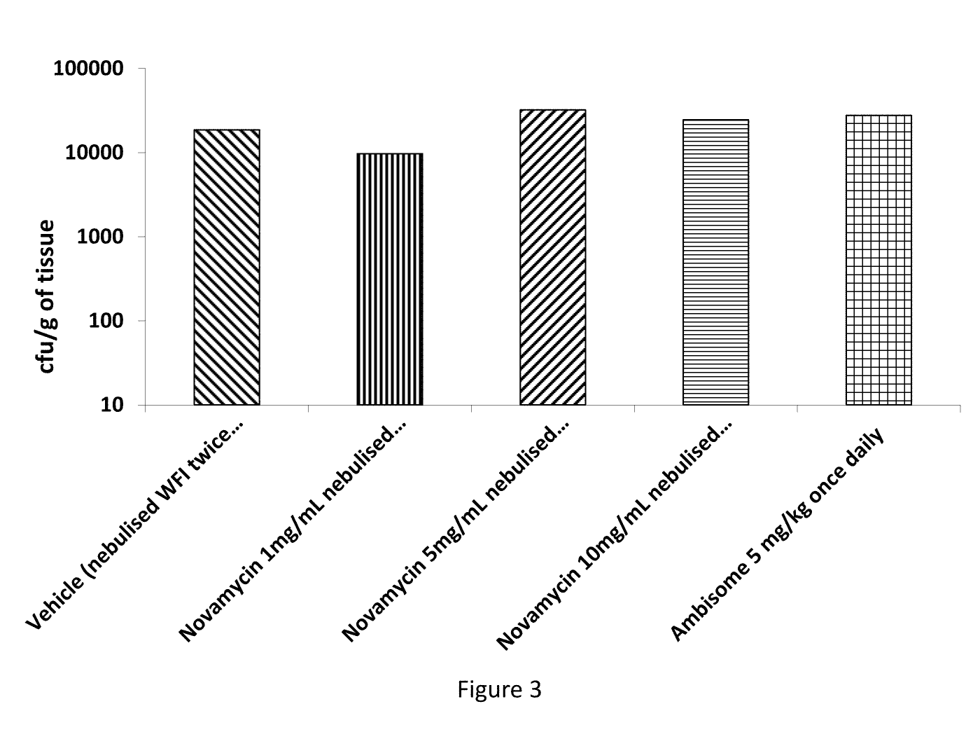

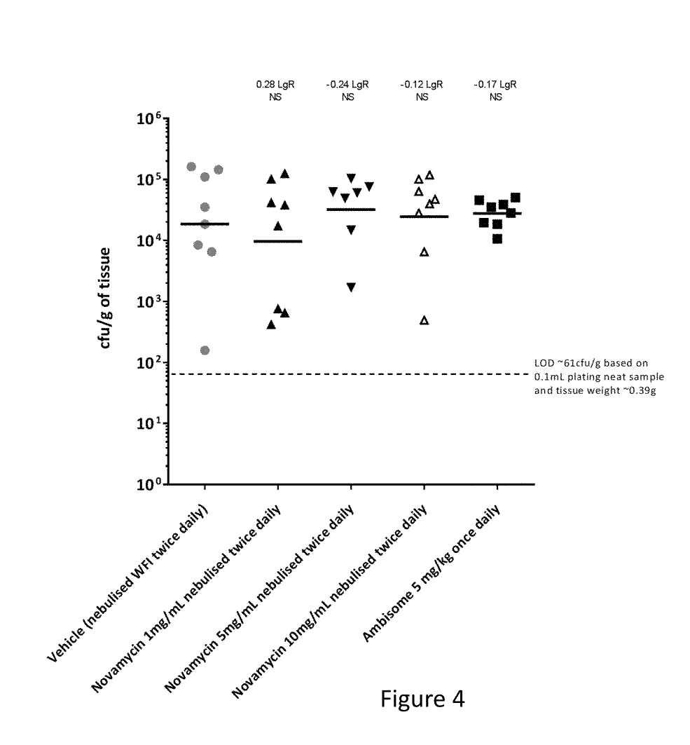

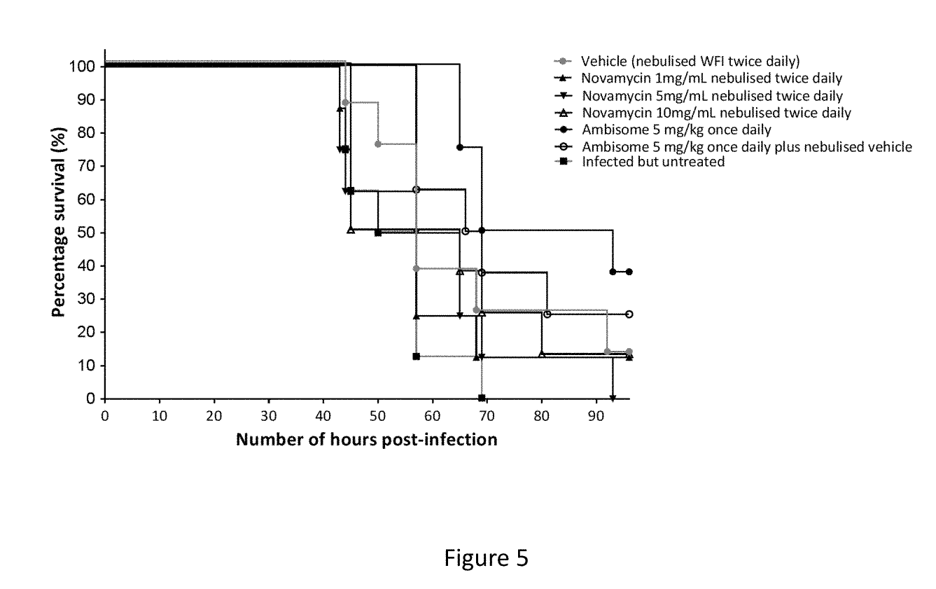

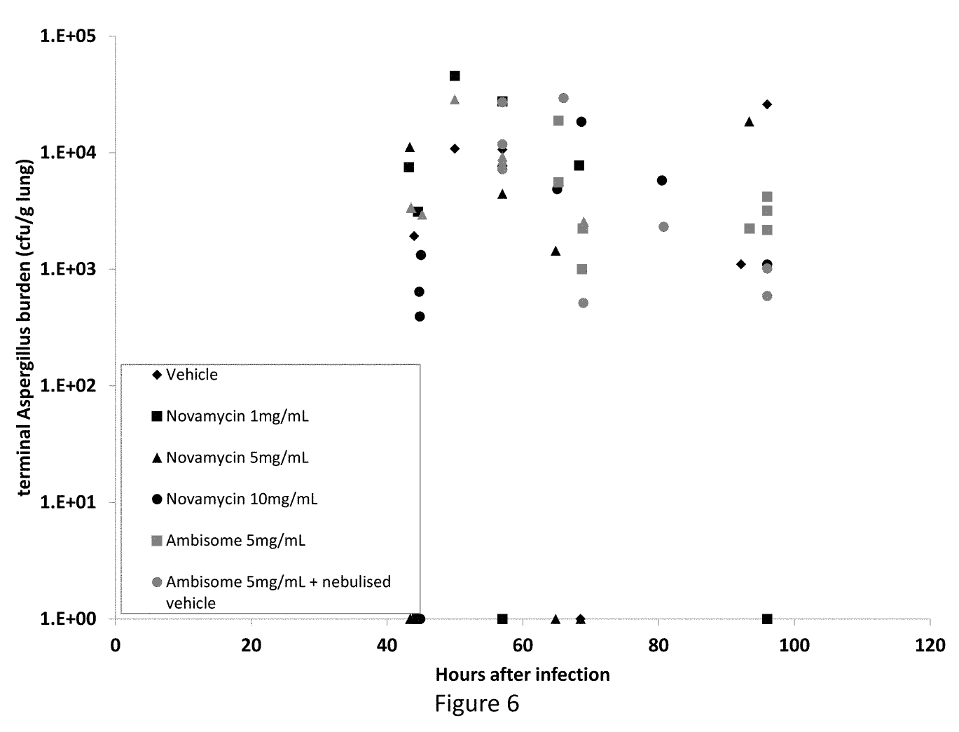

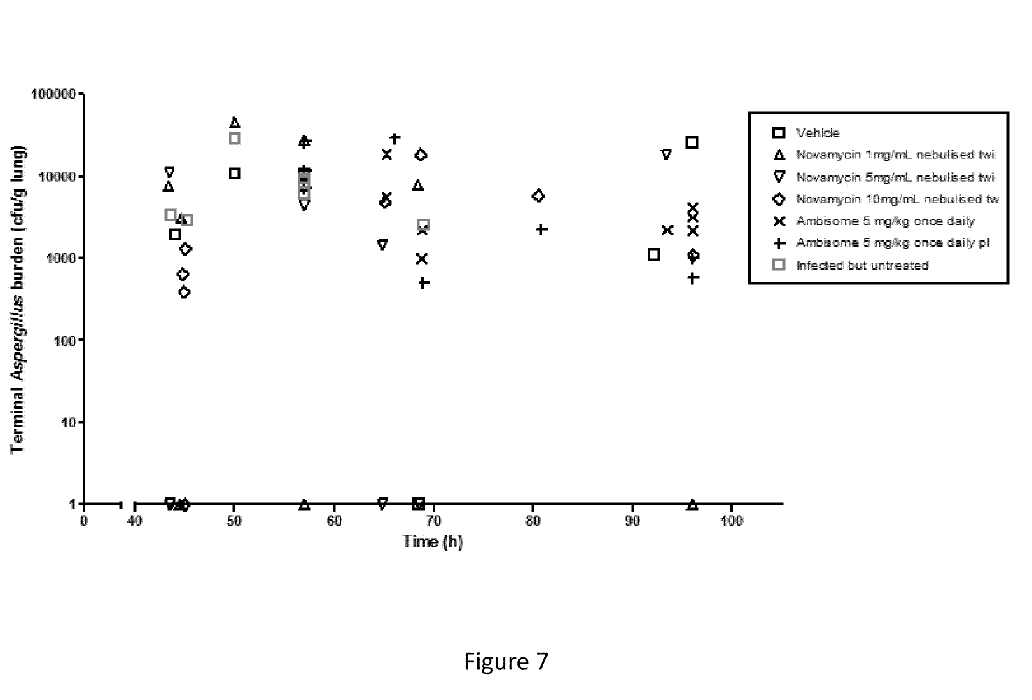

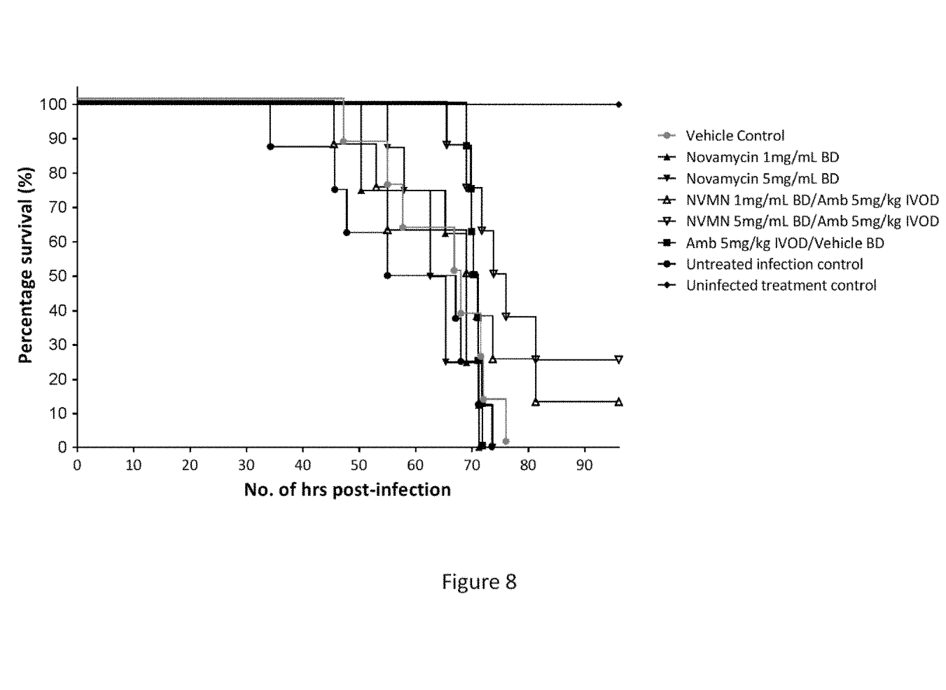

See FIGS. 1-8.

### Example 2

Antimicrobial susceptibility testing was performed based on CLSI approved standards M27-A3 and M38-A2. The effects of antimicrobial combinations were evaluated using a chequerboard method (Burkhart et al., 2006) in CLSI conditions.

In vitro studies of Aspergillus, Exophilia and Candida were performed to obtain MIC₁₀₀ results in the presence of novamycin alone and in the presence of an additional antifungal agent−AmB=Ambisome/amphotericin B, CFN=caspofungin, PCZ=pozaconazole, ITZ=itraconazole, FCZ=fluconazole.

Table 1. The MIC₁₀₀ of clinically used antifungals individually (−) and in combination (+) with Novamycin®. The MIC₁₀₀ is reduced for most antifungals when treated in combination with MIC₁₀₀ and sub-MIC₁₀₀ concentrations of Novamycin®.

(−)=MIC₁₀₀ (μg/mL) of the antifungal alone (+)=MIC₁₀₀ (μg/mL) of the antifungal in combination with Novamycin®—=Not analysed

| | MIC₁₀₀ (μg/mL) | | | | | | | | | |
|---|---|---|---|---|---|---|---|---|---|---|
| | AmB | | CFN | | PCZ | | ITZ | | FCZ | |
| | − | + | − | + | − | + | − | + | − | + |
| A. fumigatus | 0.25 | 0.03 | 0.06 | 0.06 | 0.25 | 0.125 | — | — | — | — |
| E. dermatitidis | 1 | 0.12 | 31.25 | 3.94 | — | — | 4 | 2 | >256 | >256 |
| C. albicans | 0.25 | 0.13 | 0.5 | 0.03 | 0.13 | 0.06 | — | — | — | — |

### Example 3

The neutral, additive or synergistic effect of combinations of novamycin plus other antifungal agents was studied.

| | | | | MIC₁₀₀ in | | |
|---|---|---|---|---|---|---|
| | | Antifungal | Novamycin | Combination | | |
| Pathogen | Antifungal | MIC₁₀₀ | MIC₁₀₀ | Novamycin | AF | Effect |
| A. fumigatus | Amphotericin B | 0.25 | 16 | 8 | 0.03 | Additive |
| | Posaconazole | 0.25 | 16 | 4 | 0.125 | Additive |
| | Voriconazole | 0.25 | 16 | 16 | 0.25 | Neutral |
| | Caspofungin | 0.06 | 16 | 8 | 0.04 | Additive |
| C. albicans | Amphotericin B | 0.5 | 2 | 1 | 0.05 | Additive |
| | Posaconazole | <0.03 | 1 | 1 | <0.03 | Neutral |
| | Caspofungin | 0.5 | 1 | 0.5 | 0.03 | Additive |

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An aqueous nebulisable composition comprising an antifungal peptide wherein the antifungal peptide is present at a concentration of 0.001 to 500 mg/ml.

2. The aqueous nebulisable composition according to claim 1 wherein the peptide is a linear peptide.

3. The aqueous nebulisable composition according to claim 1 wherein the peptide is a 5 to 15 amino acid peptide.

4. The aqueous nebulisable composition according to claim 3 wherein the peptide is a 10 to 15 amino acid peptide.

5. The aqueous nebulisable composition according to claim 1 wherein the peptide is a poly-arginine peptide.

6. The aqueous nebulisable composition according to claim 1 wherein the peptide is a 13 to 15 amino acid linear poly-arginine peptide.

7. The aqueous nebulisable composition according to claim 1 wherein the peptide is present at a concentration of 0.1 to 100 mg/ml.

8. The aqueous nebulisable composition according to claim 1 wherein the peptide is present at a concentration of 1 to 10 mg/ml.

9. The aqueous nebulisable composition according to claim 1 wherein the peptide is present at a concentration of approximately 5 mg/ml peptide.

10. The aqueous nebulisable composition according to claim 1 further comprising an antifungal agent.

11. The aqueous nebulisable composition according to claim 10 wherein the antifungal agent is cysteamine or a derivative thereof

12. A nebulised aqueous composition according to claim 1.

13. A method of treating or preventing a fungal infection in a subject comprising administering to the subject a nebulised aqueous composition according to claim 12.

14. The method according to claim 13 further comprising simultaneous, sequential or separate administration of a systemic antimicrobial agent.

15. The method according to claim 14 wherein the systemic antimicrobial agent is selected from the group consisting of polyenes selected from amphotericin B, amphotericin B lipid complex (ABCD), liposomal amphotericin B (L-AMB), and liposomal nystatin; azoles and triazoles selected from voriconazole, fluconazole, ketoconazole, itraconazole, and pozaconazole; glucan synthase inhibitors selected from caspofungin, micafungin (FK463), and V-echinocandin (LY303366); griseofulvin; allylamines selected from terbinafine; flucytosine; and peptides selected from Novamycin.

16. The method according to claim 15 wherein the systemic antimicrobial agent is ambisome/amphotericin B, caspofungin or cysteamine or a derivative thereof or Novamycin.

17. The method according to claim 13 wherein the fungal infection is selected from the group consisting of: Candida spp., Epidermophyton spp., Exophiala spp., Microsporum spp., Trichophyton spp., Tinea spp., Aspergillus spp., Blastomyces spp., Blastoschizomyces spp., Coccidioides spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp., Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., and Wangiella spp.

18. An aqueous nebulisable composition according to claim 1 or a nebulised aqueous composition according to claim 12 for use in the treatment of a local and optionally disseminated fungal infection.

19. The use according to claim 18 wherein the fungal infection is selected from the group consisting of: Candida spp., Epidermophyton spp., Exophiala spp., Microsporum spp., Trichophyton spp., Tinea spp., Aspergillus spp., Blastomyces spp., Blastoschizomyces spp., Coccidioides spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp., Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., and Wangiella spp.

20. An aqueous nebulisable composition according to claim 1 or the nebulised aqueous composition according to claim 12 for use in the treatment by inhaled administration once to four times per day, of a local and optionally disseminated fungal infection.

21. The use according to claim 20 wherein the administration is bidaily.

22. The use according to claim 20 wherein the administration is of 5 mg/ml for 10 to 30 minutes, such as for approximately 20 minutes.

23. The use according to claim 20 wherein the administration is repeated for up to 20 days, such as for up to 14 days.

24. The use according to claim 20 in combination with systemic administration of an antimicrobial agent.

25. The use according to claim 20 wherein the local infection is in the lung.

26. A kit of parts comprising a composition according to claim 1 and a systemic antimicrobial agent.

27. A process of making an aqueous composition according to claim 1 comprising the step of adding an antifungal peptide to water in the amount of 0.1 to 100 mg/ml.

28. A nebuliser comprising an aqueous composition according to claim 1.

29. A composition comprising a 14 amino acid linear poly-arginine peptide and an antifungal agent.

30. The composition of claim 29 wherein the antifungal agent is selected from the group consisting of: cysteamine, Novamycin, amphotericin B, caspofungin, pozaconazole, itraconazole and fluconazole.
